(19) United States
(12) Patent Application Publication
(10) Pub. No.: US 2012/0070749 A1
(43) Pub. Date: Mar. 22, 2012

(54) Process for the preparation of 4-fluoro-4-R-5-R'-1,3-dioxolane-2-ones

(75) Inventors: Martin Bomkamp, Hannover (DE); Jens Olschimke, Hannover (DE); Carsten Brosch, Seelze (DE); Andreas Grossmann, Sehnde (DE)

(73) Assignee: SOLVAY FLUOR GMBH, Hannover (DE)

(21) Appl. No.: 13/321,270

(22) PCT Filed: May 26, 2010
(86) PCT No.: PCT/EP2010/057281

§ 371 Date: Nov. 18, 2011

(30) Foreign Application Priority Data
May 28, 2009 (EP) 09161429.7

(51) Int. Cl. H01M 10/056; C07C 69/63; C09K 3/00; C07D 317/38

(52) U.S. Cl. 429/337; 549/229; 560/227; 252/364

(57) ABSTRACT

The present invention concerns 4-fluoro-4-R-5-R′-1,3-dioxolane-2-ones, wherein R is an alkyl group and R′ is H or a C1 to C3 alkyl group, their manufacture, solvent mixtures for lithium ion batteries containing them and conductive salt solutions for lithium ion batteries, e.g. solutions containing LiPF₆.

This application claims priority to European application No. 09161429.7 filed May 28^{th}, 2009, the whole content of this application being incorporated herein by reference for all purposes.

The invention concerns pure 4-fluoro-4-R-5-R′-1,3-dioxolane-2-ones wherein R is alkyl and R′ is H or a C1 to C3 alkyl group and a process for the manufacture of 4-fluoro-4-R-5-R′-1,3-dioxolane-2-ones wherein R is alkyl and R′ is H or a C1 to C3 alkyl group. It also concerns 4-chloro-4-R-5-R′-1,3-dioxolane-2-ones wherein R and R′ have the meaning given above, which are useful as intermediates in the process of the invention.

Japanese patent application 08-306364 discloses nonaqueous electrolytic solutions comprising cyclic fluorosubstituted carbonates. No way is given how these compounds may be obtained. DE Laid Open 1031800 discloses the manufacture of halogensubstituted cyclic carbonates (suitable as drugs or as intermediates of drugs) from carbonyl halide and hydroxyketones in the presence of a base and of a solvent. Only chlorosubstituted compounds were prepared in the examples.

F. S. Fawcett et al. describe in Journal of the American Chemical Society, vol. 84 (1962), pages 4275 to 4285, the addition of carbonyl fluoride to certain functional groups.

Fluorinated dialkylcarbonates and fluorinated alkylene carbonates are suitable as additives and solvents for lithium ion batteries.

Object of the present invention is to provide novel cyclic organic carbonates which contain a fluorine atom and which are suitable as additives or solvents for lithium ion batteries. This object is achieved by 4-fluoro-4-R-5-R′-1,3-dioxolane-2-ones, wherein R is alkyl and R′ is a H or a C1 to C3 alkyl group, especially 4-fluoro-4-methyl-1,3-dioxolane-2-one, and a specific process for their manufacture.

Accordingly, one aspect of the present invention concerns compounds of formula (I), 4-fluoro-4-R-5-R′-1,3-dioxolane-2-ones:

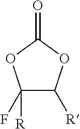

wherein R is alkyl and R′ is H or a C1 to C3 alkyl group. R preferably is C1 to C5 alkyl, more preferably, C1 to C3 alkyl. Most preferably, R denotes methyl, ethyl, i-propyl and n-propyl. A preferred embodiment of this aspect concerns pure compounds of formula (I), 4-fluoro-4-R-5-R′-1,3-dioxolane-2-ones:

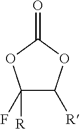

wherein R is alkyl and R′ is H or a C1 to C3 alkyl group. R preferably is C1 to C5 alkyl, more preferably, C1 to C3 alkyl. Most preferably, R denotes methyl, ethyl, i-propyl and n-propyl. The term “pure” denotes preferably a single compound of formula (I) which has a degree of purity of equal to or more than 99% by weight, more preferably, a degree of purity of equal to or more than 99.5% by weight, very preferably of equal to or more than 99.9% by weight, and especially of equal to or more than 99.99% by weight. R′ is preferably H.

Pure 4-fluoro-4-R-5-R′-1,3-dioxolane-2-one with a purity of equal to or greater than 99% by weight wherein R denotes C1 to C5 alkyl are preferred. A very preferred compound of formula (I) is 4-fluoro-4-methyl-1,3-dioxolane-2-one. 4-fluoro-4-ethyl-1,3-dioxolane-2-one, 4-fluoro-4-n-propyl-1,3-dioxolane-2-one and 4-fluoro-4-i-propyl-1,3-dioxolane-2-one are also preferred. In these compounds, R′ is H.
It has to be noted that compounds of formula (I) wherein R′ is a C1 to C3 alkyl group, exist in the form of cis and trans isomers.

The most preferred compound is 4-fluoro-4-methyl-1,3-dioxolane-2-one. In this compound, R′ is H.

Another aspect of the present invention concerns methods for the preparation of 4-fluoro-4-R-5-R′-1,3-dioxolane-2-ones wherein R is alkyl and R′ denotes H or a C1 to C3 alkyl group. R denotes preferably C1 to C5 alkyl, more preferably, C1 to C3 alkyl. The process of the invention for the preparation of 4-fluoro-4-R-5-R′-1,3-dioxolane-2-ones wherein R is alkyl and R′ is H or a C1 to C3 group comprises
a step of cyclization of compounds of formula (II), FC(O)OCHR′C(O)R wherein R is alkyl and R′ is H or a C1 to C3 group, or
it comprises a step of cyclization of compounds of formula (II′), ClC(O)OCHR′C(O)R wherein R is alkyl and R′ is H or a C1 to C3 group, and a step of a subsequent chlorine-fluorine exchange.

According to one alternative, 4-fluoro-4-R-5-R′-1,3-dioxolane-2-ones are prepared by cyclization of compounds of formula (II), FC(O)OCHR′C(O)R wherein R is alkyl and R′ is H or C1 to C3 alkyl. R denotes preferably C1 to C5 alkyl, more preferably, C1 to C3 alkyl. Most preferably, R denotes methyl, ethyl, i-propyl and n-propyl. R′ preferably is H. Especially preferably, R is methyl and R′ is H.

The cyclization reaction is preferably catalyzed.

According to one embodiment, the cyclization reaction is catalyzed by a nitrogen containing heterocyclic compound or by fluoride ions. The F⁻ ions can be introduced into the reaction mixture in the form of a salt, preferably an inorganic salt. Alkali metal fluorides, especially LiF, are preferred salts to provide F⁻ ions. In a preferred embodiment, the heterocyclic compound is an aromatic compound. For example, pyridine or 2-methylimidazole can be used as catalyst. Especially preferred are pyridines substituted by at least one dialkylamino group, especially those having a dialkylamino group in the 4-position. 4-Dimethylaminopyridine is very suitable. Other 4-dialkylaminopyridines, for example, those wherein alkyl denotes a C1 to C3 alkyl group, are also considered to be suitable. The alkyl groups can be the same or different.

The nitrogen containing heterocyclic compound can be present in the reaction mixture in a broad range. For example, it can be present in an amount of 0.1 to 10% by weight of the reaction mixture.

According to another embodiment, the cyclization reaction is catalyzed by acids, especially by hydrogen fluoride (HF). If desired, the acid catalyst can be added to the starting compound. According to a preferred embodiment, the starting compound is prepared from carbonyl chloride, carbonyl fluoride or carbonyl chloride fluoride and hydroxyketones as will be explained later. In this embodiment, acid, namely HCl and/or HF, is produced as reaction product. Thus, in this embodiment, it is not necessary to add acid to catalyze the cyclization reaction. This, of course, is advantageous because it obviates a separate step to add the acid catalyst.

The cyclization reaction is preferably performed at a temperature equal to or higher than 20° C. It is preferably performed at a temperature equal to or higher than 50° C. It is preferably performed at a temperature equal to or lower than 200° C.

The reaction is performed in the liquid phase. It can be performed batch wise or continuously.

The cyclization reaction can be performed neat or in the presence of a solvent. Suitable solvents are aprotic organic solvents. For example, ethers, esters, chlorocarbons, perfluorocarbons, chlorofluorocarbons, perfluorocarbons, hydrochlorocarbons, hydrocarbons, and aromatic hydrocarbons, for example, benzene, benzene substituted by one or more C1 to C3 alkyl groups, benzene substituted by one or more halogen atoms, are suitable. Toluene or tetrahydrofuran are very suitable. Also the respective target product, the 4-fluoro-4-alkyl-5-R′-1,3-dioxolane-2-one is a suitable solvent; workup is especially easy because no additional compound must be separated.

The produced 4-fluoro-4-R-5-R′-1,3-dioxolane-2-one can be isolated in a known manner, e.g. by distillation, crystallization or precipitation.

In a preferred embodiment, the compounds of formula (II) are prepared from carbonyl fluoride or carbonyl chloride fluoride and hydroxyketones of formula (III), RC(O)CHR′OH wherein R is alkyl, and wherein R′ denotes H or a C1 to C3 alkyl group. R preferably is a C1 to C5 alkyl group, more preferably, a C1 to C3 alkyl group. Most preferably, R denotes methyl, ethyl, i-propyl and n-propyl. R′ is preferably H. Especially preferably, a compound of formula (III) is used as starting material wherein R is methyl, ethyl, i-propyl or n-propyl, and R′ is H.

The molar ratio between carbonyl fluoride or carbonyl chloride fluoride and the hydroxyketone preferably is equal to or greater than 0.95:1, more preferably, equal to or greater than 1:1. It is preferably equal to or lower than 4:1; more preferably, it is equal to or lower than 2.5:1. A slight molar excess of carbonyl fluoride or carbonyl chloride fluoride is advantageous.

According to one preferred alternative, the reaction between carbonyl fluoride and hydroxyacetone is preferably performed in the presence of an HF scavenger, for example, in the presence of a tertiary amine, a fluoride salt which absorbs HF, or an N-heterocyclic aromatic compound. Especially preferably, LiF, NaF, KF or CsF are applied as HF scavenger.

According to one preferred alternative, the reaction between carbonyl chloride fluoride and hydroxyacetone is performed in the presence of an HCl scavenger, for example, in the presence of a tertiary amine or an N-heterocyclic aromatic compound. Any resulting HF will also be bound by the scavenger.

The reaction between the carbonyl compound and the ketone is preferably performed in the liquid phase. It is preferably performed at a temperature equal to or lower than 50° C. More preferably, it is performed at a temperature equal to or lower than 0° C.

The reaction can be performed neat or in the presence of a solvent. Suitable solvents are aprotic organic solvents. For example, ethers, esters, chlorocarbons, perfluorocarbons, chlorofluorocarbons-, perfluorocarbons, hydrochlorocarbons, hydrocarbons, and aromatic hydrocarbons, for example, benzene, benzene substituted by one or more C1 to C3 alkyl groups, benzene substituted by one or more halogen atoms, are suitable. Toluene or tetrahydrofuran are very suitable. The respective compound of formula (I) for which the reaction product between the compound of formula (II) and hydroxyketone is used as intermediate can also be used as solvent.

If desired, the compound of formula (II) can be isolated by known methods, e.g., by distillation, crystallization or precipitation. Preferably, it is further reacted in a second step to the compounds of formula (I), without isolation, as described above.

According to a second preferred alternative, the reaction between carbonyl fluoride or carbonyl chloride fluoride and the hydroxyketone is performed in the absence of an acid scavenger. In this alternative, it is preferred to perform the reaction in the absence of a solvent.

It was found that the carbonyl compound and the hydroxyketone react in a one-pot reaction to form the compound of formula (I). The intermediate compound of formula (II) must not be isolated or purified. Accordingly, a preferred embodiment provides a method for the preparation of compounds of formula (I) wherein the compound of formula (II), FC(O)OCHR′C(O)R wherein R and R′ have the meaning given above, is prepared in a first step from carbonyl fluoride or carbonyl chloride fluoride and a hydroxyacetone of formula (III), RC(O)CHR′OH wherein R and R′ have the meaning given above, and wherein the compound of formula (II) formed in the first step is further reacted in a second step to form the compound of formula (I) in which method the first step and the second step are performed in a one-pot reaction. Preferably, when the reaction is started, the mixture of starting compounds consists of the carbonyl compound and the hydroxyketone. Since HF is released during the reaction if carbonyl fluoride is used as carbonyl compound, or HCl and HF are released if carbonyl chloride fluoride is used as carbonyl compound, the reaction mixture comprises HF and/or HC after the reaction has started. This is different to reactions which are performed in the presence of bases like tertiary amines because these bases bind formed hydrogen halide.

No isolation of the intermediate compound of formula (II) is necessary. The HF or HCl which are produced as by-product in the reaction between the carbonyl halide and the hydroxyketone can be left in the reaction mixture, or they can be removed during the reaction or after it has terminated. The acid, especially HF, seem to act as a catalyst.

In this embodiment, carbonyl fluoride is the preferred carbonyl halide. If the removal of HF formed is not intended during the reaction, the starting materials are given into a cooled reactor, especially a pressurizable reactor. Cooling of the reactor is stopped, and the reaction mixture is brought to room temperature, by warming the reactor content or by letting the temperature rise to room temperature. While the compound of formula (I) is formed even at room temperature (about 20° C.), it is preferred to heat the reaction mixture. Preferably, the reaction mixture is heated to a temperature which is equal to or lower than 70° C. Preferably, the reaction mixture is reacted under autogenous pressure in an autoclave. It is preferred to stir the reaction mixture or to apply other means for mixture the reactor content.

The resulting HCl and HF (when carbonyl chloride fluoride is used as starting compound) or, in the preferred embodiment wherein carbonyl fluoride is used, the resulting HF is removed from the reaction mixture after termination of the reaction. The invention will now be explained further in view of the preferred embodiment which uses carbonyl fluoride as starting compound.

If an autoclave was applied as reactor, the pressure is released. Then, hydrogen fluoride is removed from the reaction mixture by methods known in the art. For example, the reaction mixture is distilled, or a vacuum is applied. A preferred way to remove HF is to pass inert gas through the reaction mixture. Nitrogen is especially suitable as inert gas. The reaction mixture and/or the inert gas can be heated to improve the removal of HF. A vacuum can be applied while passing inert gas through the reaction mixture to improve or speed up the HF removal. If the HF content in the resulting raw product has the desired low level, for example, if the HF content is equal to or lower than 2% by weight in the resulting raw product, the resulting raw product which essentially contains the compound of formula (I), may be subjected to at least one further purification step. The further purification step or steps can be chromatographic methods. It is preferred to purify the raw compound of formula (I) by distillation.

If it is intended to remove the hydrogen halide (HF when carbonyl fluoride is used as the starting material, HF and HCl if carbonyl chloride fluoride is used as starting material), it is preferred to perform the reaction such that an inert gas, especially nitrogen, is passed through the reaction mixture to remove at least a part of the hydrogen halide formed during the reaction. The reaction is preferably performed at ambient pressure. No acid scavenger is applied, i.e. hydrogen halide is present in the reaction mixture, and preferably, the reaction is performed in the absence of a solvent, i.e. solventless. The carbonyl fluoride can be added to the hydroxyketone before starting the reaction; in an alternative way of performing the reaction, carbonyl fluoride and inert gas, especially nitrogen, are passed continuously through the liquid in the reactor. Carbonyl compound and inert gas can be entered separately into the reactor, or in the form of a mixture. The volume ratio of carbonyl compound and inert gas can vary, for example, in a range of 1:9 to 9:1. Another aspect of the present invention are compounds of formula (II), FC(O)OCHR′C(O)R wherein R is alkyl, and wherein R′ is H or a C1 to C3 alkyl. The term “alkyl” in relation to the substituent on the C4 atom denotes preferably C1 to C5 alkyl, more preferably, C1 to C3 alkyl. Most preferably, it denotes methyl, ethyl, i-propyl and n-propyl. Especially preferably, R is methyl, and R′ is H. These compounds can be manufactured as described above, and they can be used as intermediates to prepare compounds of formula (I) which are useful as additives or solvents of lithium ion batteries, as described above.

In the following, another alternative for the manufacture of the compounds of formula (I) is described.

A preferred process of the invention comprises
a) a step of reacting phosgene, diphosgene or triphosgene and a hydroxyacetone of formula (III), RC(O)CHR′OH wherein R is an alkyl group and R′ is H or a C1 to C3 alkyl group to form a reaction mixture containing ClC(O)OCHR′C(O)R,
b) a step of performing a cyclization reaction, and

a step of performing a chlorine-fluorine exchange reaction,
wherein the chlorine-fluorine exchange reaction is performed after step a) so that a reaction mixture is formed containing FC(O)OCHR′C(O)R which, after optional purification, is subjected to the cyclization reaction of step b), or wherein the chlorine-fluorine exchange reaction is performed after step b) so that ClC(O)OCHR′C(O)R formed in step a) is converted to 4-chloro-4-R-5-R′-1,3-dioxolane-2-one which then is subjected to the chlorine-fluorine exchange reaction to form 4-fluoro-4-R-5-R′-1,3-dioxolane-2-one,
with the proviso that R is an alkyl group and R′ is H or a C1 to C3 alkyl group. Of course, R and R′ of the starting material have the same meaning as R and R′ in the intermediate product and the final product.

In this alternative, chlorosubstituted compounds are involved, and the compounds of formula (II) are prepared by a chlorine-fluorine exchange reaction from the respective compounds of formula (IV), 4-chloro-4-R-5-R′-1,3-dioxolane-2-ones:

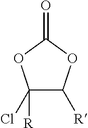

Wherein R is alkyl and R′ is H or a C1 to C3 alkyl group. R preferably is a C1 to C5 alkyl group, more preferably, a C1 to C3 alkyl group. Most preferably, R denotes methyl, ethyl, i-propyl and n-propyl.

The compounds of formula (IV), 4-chloro-4-R-5-R′-1,3-dioxolane-2-one wherein R is an alkyl group and R′ is H, preferably pure compounds of formula (IV), 4-chloro-4-R-5-R′-1,3-dioxolane-2-one wherein R is an alkyl group and R′ is H having a purity of equal to or greater than 99% by weight, more preferably having a purity of equal to or greater than 99.9% by weight, are novel and also an embodiment of the present invention. Preferred compound is 4-chloro-4-methyl -1,3-dioxolane-2-one. The compounds of formula (IV) are useful as intermediate to prepare compounds of formula (I), for example, in the manner as described below.

The intermediate chloro substituted carbonate of formula (IV) is reacted with a reactant capable of substituting a fluorine atom for the chlorine atom. This reaction is known as “Halex” reaction. Reactants suitable to perform a chlorine-fluorine exchange are generally known. Especially suitable as such a reactant are alkaline or alkaline earth metal fluorides, ammonium fluoride, amine hydrofluorides of formula (VI), N(R¹)₄ wherein the substituents R¹ are the same or different and denote H or C1 to C5 groups with the proviso that at least 1 substituent R′ is a C1 to C5 alkyl group. Instead of the fluorides, or additionally to them, hydrofluoride adducts can be used for the Halex reaction, e.g. CsF.HF. Other fluorides are likewise suitable as reactant, e.g. AgF. The Halex reaction can be performed in the absence or in the presence of a solvent, for example, in the presence of a nitrile or an ether. Often, the reaction is performed at elevated temperature, e.g. at a temperature equal to or higher than 50° C.

The workup of the reaction mixture which comprises the chloride salt and possibly excess fluoride salt of the fluorinating reactant, and the fluorinated carbonate and possibly unreacted starting material, is performed in a known manner. For example, solids are removed by filtration, and the liquid phase is subjected to an aqueous extraction and a fractionated distillation or precipitation after removal of any solvents.

The compounds of formula (IV) are preferably manufactured by reacting carbonyl chloride or its dimer (diphosgene) or trimer (triphosgene) with hydroxyketones of formula (III), RC(O)CHR′OH wherein R is alkyl, R′ is H or a C1 to C3 alkyl group to form of compounds of formula (II′), ClC(O)OCHR′C(O)R wherein R and R′ have the meaning given above, and performing a subsequent cyclization reaction. Most preferably, R denotes methyl, ethyl, i-propyl or n-propyl. R′ is preferably H. Especially preferably, a compound of formula (III) is used as starting material wherein R is methyl, ethyl, i-propyl or n-propyl, and R′ is H. The conditions for performing the reaction between phosgene or its dimer or trimer and the hydroxyketone are as described for the respective reaction between carbonyl fluoride and the hydroxyketone. The conditions for the cyclization reaction correspond to those as described above for the cyclization reaction of the respective compounds of formula (II). The cyclic product is then reacted with a fluorinating reagent, as described above, to give a compound of formula (I).

According to a modification of this embodiment, in a first step, carbonyl chloride or its dimer (diphosgene) or trimer (triphosgene) with hydroxyketones of formula (III), RC(O)CHR′OH, wherein R is alkyl, R′ is H or a C1 to C3 alkyl group to form of compounds of formula (II′), ClC(O)OCHR′C(O)R, wherein R and R′ have the meaning given above. The resulting chlorocompound is then subjected to the corresponding fluorocompound by a Halex reaction as described above. The resulting FC(O)OCHR′C(O)R is then subjected to a cyclization reaction as described above.

The preferred process for the manufacture of compounds of formula (I), 4-fluoro-4-R-5-R′-1,3-dioxolane-2-ones, comprises:
a step of reacting carbonyl fluoride and hydroxyketones of formula (III), RC(O)CHR′OH wherein R is alkyl and preferably denotes C1 to C5 alkyl, more preferably, C1 to C3 alkyl, and most preferably, denotes methyl, ethyl, i-propyl and n-propyl, and R′ denotes H or a C1 to C3 alkyl group, to produce compounds of formula (II), FC(O)OCHR′C(O)R wherein R and R′ have the meaning given above, and
a step of cyclization of compounds of formula (II) to produce 4-fluoro-4-R-5-R′-1,3-dioxolane-2-ones.

Preferred reaction conditions of the 2 steps are explained in detail above.

It has to be noted that all products are or may be obtained as a racemic mixture of enantiomers and in addition in the case of compounds of formula (I) wherein R′ is a C1 to C3 alkyl group, a mixture of diastereomeric cis and trans isomers are obtained. It also has to be noted that the term “pure compound” includes the racemic mixtures of enantiomers. The term “pure compound” also includes the mixture of diastereomeric cis and trans isomers.

The 4-fluoro-4-R-5-R′-1,3-dioxolane-2-ones, wherein R is alkyl and preferably denotes C1 to C5 alkyl, more preferably, C1 to C3 alkyl, and most preferably, denotes methyl, ethyl, i-propyl and n-propyl, and R′ denotes H or a C1 to C3 alkyl group, notably 4-fluoro-4-methyl-1,3-dioxolane-2-one, of the present invention are especially useful as solvents or additives for lithium ion batteries. While they could be used as neat solvents, it is preferred to apply them as an additive together with one or more solvents which are known as suitable solvent or solvents for lithium ion batteries. The compounds of the invention are assumed to form a protective film on at least one of the electrodes, presumably on the anode.

Consequently, solvent mixtures containing at least one 4-fluoro-4-R-5-R′-1,3-dioxolane-2-one wherein R is alkyl and preferably denotes C1 to C5 alkyl, more preferably, C1 to C3 alkyl, and most preferably, denotes methyl, ethyl, i-propyl and n-propyl, and R′ denotes H or a C1 to C3 alkyl group, and at least one other solvent suitable for lithium ion batteries, are still another object of the present invention.

The at least one other solvent of the solvent mixture is any solvent known to be useful as solvent for Li ion batteries; it is preferably selected from the group consisting of dialkyl carbonates and alkylene carbonates, preferably from the group consisting of dimethyl carbonate, diethyl carbonate, ethyl methyl carbonate, ethylene carbonate and propylene carbonate.

If they are used mainly in their function as solvents, the 4-fluoro-4-R-5-R′-1,3-dioxolane-2-one can constitute 100% by weight of the solvents, or their content in a mixture with other solvents used in lithium ion batteries, e.g. those mentioned above, can be quite high, e.g. 20% by weight up to <100% by weight of the solvent mixture.

If they are contained mainly in their function as additives in a mixture with solvents, e.g. for providing a protective film on at least one of the electrodes of the battery, they preferably are contained in the mixture with the solvent or solvents in amount of equal to or greater than 0.5% by weight of the total weight of the mixture. Preferably, they are contained in the mixture with the solvent or solvents in an amount equal to or lower than 20% by weight of the mixture. Often, their content is equal to or lower than 10% by weight in the mixture.

Preferably, the solvent mixture for lithium ion batteries contains a fluorinated additive and at least 1 further lithium ion battery solvent with the proviso that the at least one fluorinated additive is a single, pure compound of formula (I), 4-fluoro-4-R-5-R′-1,3-dioxolane-2-one, wherein R is alkyl and R′ is H or a C1 to C3 alkyl group and with the proviso that the purity of the compound of formula (I) is equal to or greater than 99.9% by weight.

If desired, the mixture of solvent and additive may contain further additives, for example, fluoroethylene carbonate, tert-amylbenzene or tris(2,2,2-trifluoroethyl)phosphate.

Still another aspect of the present invention concerns electrolyte solutions for lithium ion batteries. These electrolyte solutions contain the mixture of additive and solvent described above, and a conducting salt. The conducting salt is known in the art. LiPF₆ is the preferred a conducting salt. Other conducting salts are also suitable as constituent of the electrolyte solutions of the present invention, for example, e.g. Lithium bisoxalatoborate (LiBOB), Lithium bis(fluorosulfonyl)imide (LiFSI), Lithium bis(trifluorsulfonyl)imide (LiTFSI) or LiBF₄.

While the amount of conducting salt in the electrolyte solution is variable, usually 1±0.5 mol of the conducting salt is contained in dissolved form.

The process of the present invention allows the selective manufacture of high purity dioxolanones which contain selectively a fluorine atom on the C4 carbon atom of the ring. Thus, it is easily possible to produce a solvent or solvent mixture for lithium ion batteries with defined properties.

Should the disclosure of any patents, patent applications, and publications which are incorporated herein by reference conflict with the description of the present application to the extent that it may render a term unclear, the present description shall take precedence.

The invention will now be described by examples without the intention to limit it.

### EXAMPLE 1

### Preparation of FC(O)OCH₂C(O)CH₃

Hydroxyacetone (31.74 g; 0.42 mol; available from Alfa Aeser) was dissolved in 50 ml of dried toluene. Dried NaF (32 g, 0.76 mol) was added as HF scavenger. During 2 hours, carbonyl fluoride (45g; 0.68 mol) was introduced into the solution which was kept at −78° C. The resulting reaction mixture was then brought to ambient temperature (about 20° C.), and the solid (mainly NaF.HF) was filtered off.

If desired, the produced FC(O)OCH₂C(O)CH₃ can be isolated by fractionated distillation.

### EXAMPLE 2

### Preparation of 4-fluoro-4-methyl-1,3-dioxolane-2-one

To the filtered solution of example4-(Dimethylamino)pyridine (1.5 g) was added, and the resulting solution was stirred for 4 hours at 80° C. After cooling, volatile constituents, especially toluene, were removed by a rotating evaporator, and 4-fluoro-4-methyl-1,3-dioxolane-2-one was isolated by fractionated distillation.

The boiling point was 88° C. (20 mbar).
Yield: 11.72 g (21% of the theory)

### EXAMPLE 3

### Preparation of a Solvent Mixture for Lithium Ion Batteries

3.1. Mixture containing 4-fluoro-4-methyl-1,3-dioxolane-2-one and ethylene carbonate

4-fluoro-4-methyl-1,3-dioxolane-2-one and ethylene carbonate are mixed in a weight ratio of 1:19.
3.2. Mixture containing 4-fluoro-4-methyl-1,3-dioxolane-2-one and dimethyl carbonate

4-fluoro-4-methyl-1,3-dioxolane-2-one and dimethyl carbonate are mixed in a weight ratio of 1:19.
3.3. Mixture containing 4-fluoro-4-methyl-1,3-dioxolane-2-one and propylene carbonate

4-fluoro-4-methyl-1,3-dioxolane-2-one and propylene carbonate are mixed in a weight ratio of 1:19.
3.4. Mixture containing 4-fluoro-4-methyl-1,3-dioxolane-2-one, ethylene carbonate and dimethyl carbonate

4-fluoro-4-methyl-1,3-dioxolane-2-one, ethylene carbonate and dimethyl carbonate are mixed in a weight ratio of 1:9.5:9.5.
3.5. Mixture containing 4-fluoro-4-methyl-1,3-dioxolane-2-one, ethylene carbonate and ethyl methyl carbonate

4-fluoro-4-methyl-1,3-dioxolane-2-one, ethylene carbonate and ethyl methyl carbonate are mixed in a weight ratio of 1:9.5:9.5.

### EXAMPLE 4

### Preparation of an Electrolyte Solution Containing LiPF₆

### EXAMPLE 4.1

### LiPF₆ Dissolved in a Solvent Mixture of Example 3.1

LiPF₆ is dissolved in the solvent mixture of example 3.1 such that the concentration of LiPF₆ is 1 molar under precautions which prevent any contact with moisture, e.g. in a glove box under an argon or nitrogen atmosphere.

### EXAMPLE 4.2

### LiPF₆ Dissolved in a Solvent Mixture of Example 3.2

LiPF₆ is dissolved in the solvent mixture of example 3.2 such that the concentration of LiPF₆ is 1 molar.

### EXAMPLE 4.3

### LiPF₆ Dissolved in a Solvent Mixture of Example 3.3

LiPF₆ is dissolved in the solvent mixture of example 3.3 such that the concentration of LiPF₆ is 1 molar.

### EXAMPLE 4.4

### LiPF₆ Dissolved in a Solvent Mixture of Example 3.4

LiPF₆ is dissolved in the solvent mixture of example 3.3 such that the concentration of LiPF₆ is 1 molar.

### EXAMPLE 5

### Preparation of FC(O)OCH₂C(O)CH₃ in the Absence of an Acid Scavenger and in the Absence of a Solvent at Ambient Pressure

Hydroxyacetone (100 g, 1.35 mol) is given into a two-necked PFA flask. Upon cooling to 0° C. by an ice/water bath a mixture of COF₂ and N₂ is bubbled through the liquid under intensive stirring until all hydroxyacetone is consumed. Now the reaction mixture is brought to 100° C. and nitrogen is bubbled through the reaction mixture for 2 h.

If desired, the produced FC(O)OCH₂C(O)CH₃ can be isolated by fractionated distillation. Alternatively, base, e.g. dimethyl aminopyridine or HF can be added and the cyclization reaction can be performed to obtain 4-fluoro-4-methyl-1,3-dioxolane-2-one.

### EXAMPLE 6

### Preparation of 4-fluoro-4-methyl-1,3-dioxolane-2-one Under Autogenous Pressure in the Absence of a Base

Hydroxyacetone (100 g, 1.35 mol) was given into a stainless steel pressure reactor. The reactor was closed and cooled in a isopropanol dry ice bath for 30 minutes. Carbonylfluoride (90 g, 1.35 mol) given to the reactor. The pressure went up to 25 bar. The reactor was kept in the isopropanol/dry ice bath for another 2 h after which the reaction was allowed to warm up to room temperature. The reaction was then heated to 50° C. for 18 h. Excess COF₂ was removed by release of pressure after which the reaction mixture was brought to 100° C. Volatile compounds were removed by stripping the mixture with nitrogen for 2 h at 100° C. The reactor was opened and the crude product was obtained as a dark viscous liquid (109.2 g) with a purity of 94.6%. If desired, the produced 4-fluoro-4-methyl-1,3-dioxolan-2-one can be isolated by fractionated distillation.

1. A compound of formula (I), 4-fluoro-4-R-5-R′-1,3-dioxolane-2-one wherein R is an alkyl group and R′ is H or a C1 to C3 alkyl group.

2. The compound of formula (I) according to claim 1, having a purity of equal to or greater than 99% by weight.

3. The compound of formula (I) according to claim 1 wherein R is an alkyl group selected from the group consisting of methyl, ethyl, i-propyl and n-propyl.

4. A process for the preparation of 4-fluoro-4-R-5-R′-1,3-dioxolane-2-one wherein R is an alkyl group and R′ is H or a C1 to C3 alkyl group, said process comprising a step of cyclization of compounds of formula (II): FC(O)OCHR′C(O)R wherein R is alkyl and R′ is H or a C1 to C3 alkyl group, or comprising a step of cyclization of compounds of formula (II′): ClC(O)OCHR′C(O)R wherein R is alkyl and R′ is H or a C1 to C3 alkyl group and a step of subsequent chlorine-fluorine exchange.

5. The process of claim 4 wherein R is a C1 to C5 alkyl group.

6. The process of claim 4 wherein the cyclization reaction is catalyzed by a nitrogen containing heterocyclic compound or F⁻.

7. The process of claim 4 wherein the compound of formula (II): FC(O)OCHR′C(O)R wherein R is an alkyl group and R′ is H or a C1 to C3 alkyl group, is prepared from carbonyl fluoride or carbonyl chloride fluoride and a hydroxyacetone of formula (III): RC(O)CHR′OH wherein R is an alkyl group and R′ is H or a C1 to C3 alkyl group.

8. The process of claim 4 comprising
a) a step of reacting phosgene, diphosgene or triphosgene and a hydroxyacetone of formula (III): RC(O)CHR′OH wherein R is an alkyl group and R′ is H or a C1 to C3 alkyl group to form a reaction mixture containing ClC(O)OCHR′C(O)R,
b) a step of performing a cyclization reaction, and
a step of performing a chlorine-fluorine exchange reaction,
wherein the chlorine-fluorine exchange reaction is performed after step a) so that a reaction mixture is formed containing FC(O)OCHR′C(O)R which, after optional purification, is subjected to the cyclization reaction of step b), or
wherein the chlorine-fluorine exchange reaction is performed after step b) so that ClC(O)OCHR′C(O)R formed in step a) is converted to 4-chloro-4-R-5-R′-1,3-dioxolane-2-one which then is subjected to the chlorine-fluorine exchange reaction to form 4-fluoro-4-R-5-R′-1,3-dioxolane-2-one,
with the proviso that R is an alkyl group and R′ is H or a C1 to C3 alkyl group.

9. The process of claim 7 wherein the compound of formula (II): FC(O)OCHR′C(O)R wherein R is an alkyl group and R′ is H or a C1 to C3 alkyl group, is prepared in a first step from carbonyl fluoride and a hydroxyacetone of formula (III): RC(O)CHR′OH wherein R is an alkyl group and R′ is H or a C1 to C3 alkyl group, wherein the compound of formula (II) formed in the first step is further reacted in a second step to form the compound of formula (I), and wherein the first step and the second step are performed in a one-pot reaction.

10. A compound of formula (II): FC(O)OCHR′C(O)R wherein R is an alkyl group, and R′ is H or a C1 to C3 alkyl group.

11. The compound of formula (II) according to claim 10 wherein the alkyl group R is a C1 to C5 alkyl group.

12. A solvent mixture for lithium ion batteries, containing at least one 4-fluoro-4-R-5-R′-1,3-dioxolane-2-one wherein R is an alkyl group and R′ is H or a C1 to C3 alkyl group, and at least one other solvent suitable for lithium ion batteries.

13. An electrolyte solution for lithium ion batteries containing the solvent mixture of claim 13 and a lithium ion battery conductive salt.

14. A compound of formula (IV): 4-chloro-4-R-5-R′-1,3-dioxolane-2-one wherein R is an alkyl group and R′ is H.

15. The compound of claim 14 having a purity of equal to or greater than 99% by weight.

16. The electrolyte solution of claim 13 wherein the lithium ion battery conductive salt is LiPF₆.
